(19) United States
(12) Patent Application Publication
(10) Pub. No.: US 2026/0013769 A1
(43) Pub. Date: Jan. 15, 2026

(54) SENSOR PLACEMENT GUIDE

(71) Applicant: Covidien LP, Mansfield, MA (US)

(72) Inventors: Shao-Nung Liang, Broomfield, CO (US); Sarah L. Hayman, Boulder, CO (US); Alexander Gordon Breakstone, Boulder, CO (US)

(73) Assignee: Covidien LP, Mansfield, MA (US)

(21) Appl. No.: 19/252,641

(22) Filed: Jun. 27, 2025

Related U.S. Application Data
(60) Provisional application No. 63/670,193, filed on Jul. 12, 2024.

(51) Int. Cl. A61B 5/271; A61B 5/1455

(52) U.S. Cl. CPC A61B 5/271; A61B 5/1455

(57) ABSTRACT

The present disclosure describes example devices and techniques for appropriately positioning a medical sensor to monitor a physiological parameter of a patient. A guide for placement of a medical sensor onto a patient includes a template having an upper edge, a lower edge, and a recess defined in the upper edge of the template. The recess includes a contour that matches a shape of a medical sensor such that the recess is sized to seat the medical sensor into the recess. The recess is displaced above the lower edge of the template by a defined distance corresponding to a clinical offset of the medical sensor above the patient's eyebrow.

### CROSS-REFERENCE TO RELATED APPLICATIONS

The present application claims benefit of priority to U.S. Provisional Patent Application No. 63/670,193, titled “Sensor Placement Guide,” and filed on Jul. 12, 2024, the disclosure of which is incorporated by reference herein in its entirety.

### FIELD

The present technology is generally related to devices and methods for positioning a medical sensor onto a patient for monitoring of a physiological parameter.

### BACKGROUND

Medical sensors are used to monitor one or more physiological parameters of a patient. For example, an oxygen saturation sensor or oximetry sensor, such as a pulse oximetry sensor or a regional oximetry sensor, can monitor the oxygen saturation levels of a patient. In some examples, a noninvasive pulse oximetry sensor is placed on a patient to measure the oxygen saturation level of the patient via photoplethysmography. When the oxygen saturation level of the patient decreases to reach a desaturation threshold, the oxygen saturation monitoring system may output an indication that the patient is experiencing oxygen desaturation, prompting a medical caregiver to take appropriate action.

### SUMMARY

The present disclosure describes example devices and techniques for appropriately positioning a medical sensor to monitor a physiological parameter of a patient.

In an embodiment, a guide for placement of a medical sensor onto a patient includes a template having an upper edge, a lower edge, and a recess defined in the upper edge of the template. The recess includes a contour that matches a shape of a medical sensor such that the recess is sized to seat the medical sensor into the recess. The recess is displaced above the lower edge of the template by a defined distance corresponding to a clinical offset of the medical sensor above the patient's eyebrow.

In an embodiment, a kit for placement of at least one medical sensor includes a package having an outer surface, a template including an upper edge, a lower edge, and at least one recess defined in the upper edge and including a contour. The kit further includes a medical sensor inside the package, at least a portion of the medical sensor having a shape that matches the contour.

In an embodiment, a guide for placement of a medical sensor onto a patient includes a template comprising an upper edge and a lower edge, first and second recesses defined in the upper edge of the template and mirror-imaged with each other. The first and second recesses each include a contour that matches a shape of a medical sensor, wherein the first recess is displaced above the lower edge of the template by a first distance and wherein the second recess is displaced above the lower edge of the template by a second distance.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is an exemplary schematic of a medical sensor, in accordance with embodiments described herein.

FIG. 2 is an exemplary schematic of an additional example of a medical sensor, in accordance with embodiments described herein.

FIG. 3 is an exemplary schematic of a subject with a plurality of medical sensors arranged in a first orientation, in accordance with embodiments described herein.

FIG. 4 is an exemplary schematic of a subject with a plurality of medical sensors arranged in a second orientation, in accordance with embodiments described herein.

FIG. 5 illustrates a subject with a medical sensor, in accordance with embodiments described herein.

FIG. 6 is an exemplary schematic of a template for use in guiding placement of at least one medical sensor, in accordance with embodiments described herein.

FIG. 7 is an exemplary schematic of a template for use in guiding placement of at least one medical sensor, in accordance with embodiments described herein.

FIG. 8 is an exemplary schematic of a template and two medical sensors arranged onto a patient, in accordance with the embodiments described herein.

FIG. 9 illustrates two templates and two medical sensors arranged onto a patient, in accordance with the embodiments described herein.

FIG. 10 illustrates an arrangement of medical sensors onto a patient after use of a placement template, in accordance with embodiments described herein.

FIG. 11 is a flow chart illustrating a method for placing at least one medical sensor, in accordance with embodiments described herein.

FIG. 12 is an exemplary schematic of a kit for packaging at least one sensor and at least one template, in accordance with embodiments described herein.

### DETAILED DESCRIPTION

The present disclosure describes example devices and techniques for appropriately positioning a medical sensor to monitor a physiological parameter of a patient, such as oxygen saturation, heart rate, depth of consciousness, blood pressure, and/or other parameters. Obtaining an accurate or reliable measurement of these parameters may depend on correctly positioning the sensor on the patient.

For example, a medical sensor positioned on a patient may be an optical sensor placed on a patient's skin, for example the forehead. Optical sensors may be used to sense temperature, blood pressure, blood flow, heart rate, respiration, blood constituents, and/or oxygen saturation. Another type of medical sensor is an electrical sensor such as an EEG (electroencephalogram) sensor, which can sense electrical signals from the patient's heart or brain. The present disclosure describes devices and techniques with reference to regional oximeter sensors (also referred to as tissue oximeter sensors) and depth of consciousness sensors (such as bispectral index sensors). However, the present disclosure may be used with other types of sensors as well such as, but not limited to, pulse oximetry, skin temperature, and blood pressure.

A regional oximeter may be used to non-invasively monitor oxygen saturation by detecting light transmitted through the patient's skin. More particularly, light may be emitted into the patient's tissue and the reflected light can be detected. An example regional oximetry sensor includes one or more light sources (such as LED's) that emit light at particular wavelengths or wavelength ranges, and two detectors (such as photodetectors) that detect light after it has passed through the patient's skin. For example, a first detector of the detectors may be arranged closer to the one or more light sources while a second detector of the detectors is arranged further away relative to the one or more light sources, generating at least two reflection detection values. The sensor sends these detected light signals to control circuitry that determines oxygen saturation based on absorption of the emitted light. For example, the sensor may send these detected light signals to a monitor and/or processor to generate the oxygen saturation values. The regional oximetry sensor may be adhered in contact with the patient's skin, for example, as an adhesive sticker, bandage, or patch. In other examples, the oximetry sensor may be held onto the patient's skin with an additional adhesive applied with the oximetry sensor. In this way, the oxygen saturation values may be continuously monitored over time, for example, over the pendency of a procedure, and continuously updated by the processor and reported to the physician or caregiver. Additional information about the derivation of tissue oximetry values from emitted and detected light can be found in U.S. Pat. No. 9,861,317 (“Methods and systems for determining regional blood oxygen saturation”, Covidien LP).

An EEG sensor, such as a bispectral index sensor (for example, BIS™ sensors from Medtronic) may be used to measure depth of consciousness of a patient. By way of example, U.S. Pat. No. 5,458,117 (“Cerebral biopotential analysis system and method”), describes a system and method for generating a bispectral index from EEG signals. The bispectral index is an EEG-based measure that quantifies a patient's level of consciousness during anesthesia and sedation from EEG signals acquired from the scalp, forehead, or temple.

Accurate measurement of regional oximetry, depth of consciousness, and other physiological parameters depends on proper placement of sensors on the patient. For example, when measuring regional oximetry, the sensors need to be arranged in a specific position to facilitate detection of signals from the desired tissue. For example, the regional oximetry sensor may be intended for detecting perfusion in a target body tissue such as the brain or kidney. If the sensor is not arranged correctly, the sensor may detect nontarget noise signals, outside of the desired brain or body tissue. Furthermore, the depth of consciousness sensor may similarly need to be arranged accurately in order to detect EEG signals of the brain tissue effectively and accurately.

Due to the variability in the size and age of patients, it may be difficult for physicians and/or caregivers to position the various seniors onto patient's skin quickly and accurately. The process may further be complicated for the caregiver if multiple sensors and multiple types of sensors need to be arranged onto the patient (i.e., both a regional oximetry sensor and a depth of consciousness sensor). Thus, it is advantageous to provide systems and methods for the caregiver to place various sensors more easily and accurately onto a patient to improve the accuracy of the detected physiological parameters.

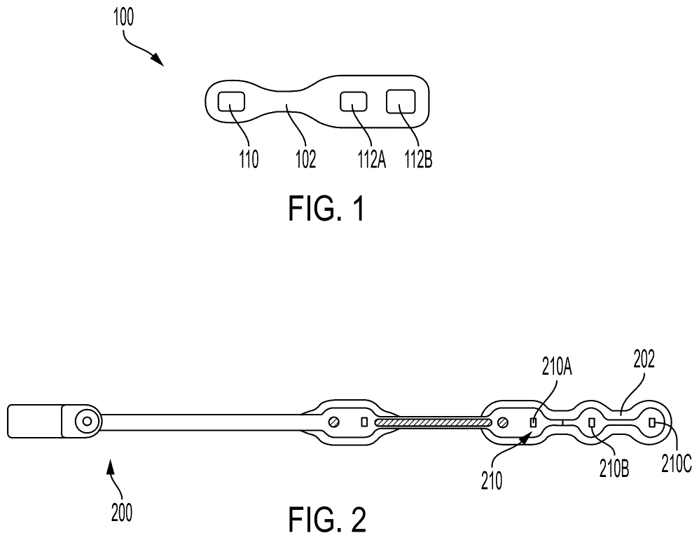

FIG. 1 illustrates an embodiment of a regional oximetry sensor 100. The sensor 100 includes a first surface, more particularly a lower surface 102, and a second surface opposite the lower surface 102, more particularly an upper surface (not shown). The lower surface 102 of the sensor 100 contacts the patient's skin. The lower surface 102 of the sensor 100 may include an adhesive material to secure the sensor 100 to the patient's skin. In other instances, additional methods of affixing the sensor 100 to the patient's skin may be incorporated, such as through external bandages, ties, or straps that may be applied to the sensor and the patient. The sensor 100 includes a light emitter 110 and at least two photodetectors, illustratively a first photodetector 112A and a second photodetector 112B. In other embodiments the sensor 100 may have additional light emitters or photodetectors.

The light emitter 110 emits light of two or more wavelengths into the patient's skin. The light is received at each of the first photodetector 112A and the second photodetector 112B after passing through the patient's skin and tissue. The sensor 100 may send the detected light signals to a monitor or processor, which uses the differential absorption of light at the first detector 112A and the second detector 112B, at multiple wavelengths such as red and infrared, to determine tissue oxygen saturation. An example regional oximetry sensor is an INVOS™ sensor available from Medtronic. However, various other regional oximetry sensors may be used.

Furthermore, the embodiments described herein may apply to various other sensors, such as a depth of consciousness sensor. For example, FIG. 2 illustrates an embodiment of a depth of consciousness sensor 200. The sensor 200 includes a first surface, illustratively a lower surface 202 that contacts the patient's skin, and a second upper surface (not shown) opposite the lower surface 202. The lower surface 202 may include an adhesive configured to attach the sensor 200 to the patient's skin. The sensor 200 further includes a set of electrodes 210. The set of electrodes 210 includes a first electrode 210A, a second electrode 210B, and a third electrode 210C. As illustrated, the electrodes 210 are arranged on the lower surface 202 of the sensor 200 such that when the sensor 200 is secured to the patient's skin, for example the patient's forehead, the electrodes 210 contact the patient's forehead. Thus, after application of the sensor 200 onto the patient, the electrodes 210 can detect electrical activity of the patient's brain. The sensor 200 receives the electrical signals from the electrodes 210 and sends the electrical signals to a monitor or processor, which uses the electrical signals to determine the depth of consciousness (or level of sedation) of the patient. In some examples, the depth consciousness sensor is a BIS™ sensor available from Medtronic. However, various other depth consciousness sensors may be used.

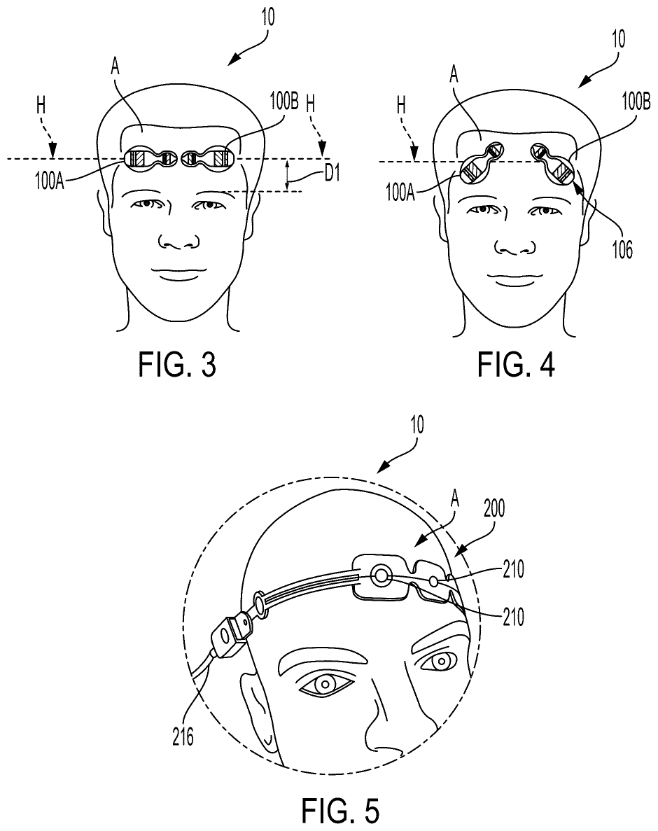

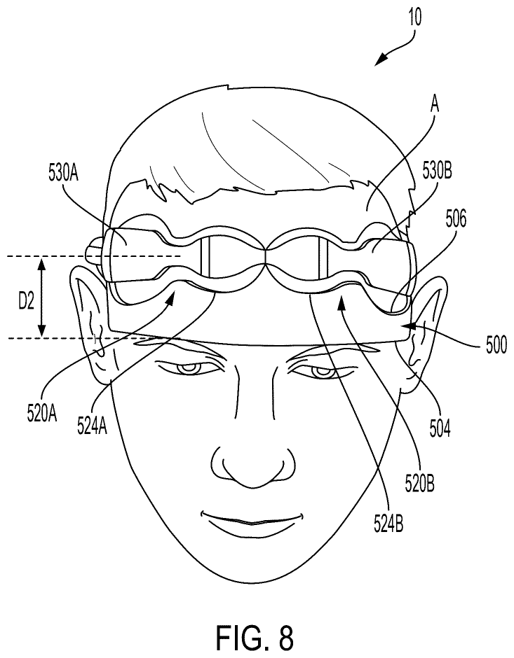

As previously recited, it is necessary that the sensors (such as the regional oximetry sensor 100 or depth of consciousness sensor 200) are arranged properly onto the skin of a patient for accurate monitoring. FIG. 3 shows an example of proper placement of two regional oximetry sensors on the patient. In this embodiment, the regional oximetry sensors are similar to, or the same as, the sensors 100 illustrated in FIG. 1. As illustrated in FIG. 3, a first regional oximetry sensor 100A and a second regional oximetry sensor 100B are arranged on a forehead A of a patient 10. The two regional oximetry sensors 100A, 100B measure oxygen saturation of the right and left hemispheres of the brain, respectively. As illustrated in FIG. 3, the sensors 100 are placed horizontally, and more particularly within plus or minus 10 degrees of a horizontal axis H across the forehead A of the patient 10, parallel with the eyebrows, symmetrically, and with a displacement D1 above the patient's eyebrows. The horizontal axis H indicates the desired location of the physiologic measurement on the patient, indicating the location where the sensing should take place. In FIG. 3, axis H corresponds to the location of brain tissue that is desired to be measured, above the sinus cavities. More particularly, the displacement D1 is defined as the distance between the top of the eyebrow of the patient 10 and the sensing location at axis H. The sensors 100A, 100B include a sensing axis along which the sensing elements are positioned on the sensor. The sensing elements include optical emitters and detectors in the case of regional oximetry, or electrodes or other sensing elements depending on the type of sensor. In FIG. 3, each sensor is placed on the patient such that the sensing axis of the sensor aligns with the axis H. In this way, the displacement D1 quantifies the distance extending between a patient's eyebrows and the sensing axis of the sensor. In some examples, the displacement D1 is at least 3 cm. In other examples, the displacement D1 is approximately 5 cm. However, various other displacement values may vary depending on the patient 10 being monitored. The displacement D will be described further with reference to FIG. 8.

With continued reference to FIG. 3, the sensors 100A, 100B are arranged in proximity to a middle point of the patient's forehead A. In some embodiments, the sensors 100A, 100B may be arranged such that they are in near contact and/or in direct contact with each other at the middle point of the patient's forehead A, above the nose. The illustrated placement of the sensors 100 is such that the sensors 100, and more particularly the one or more emitters 110 and photodetectors 112A, 112B, are arranged directly over brain tissue of the patient 10. Furthermore, the sensors 100 are positioned such that the sensors 100 do not pick up light or electrical signals from the sinus cavities above eyebrows of the patient 10. Light signals from the sinus cavities are not representative of brain oxygenation and thus proper placement of the sensors avoids placement over the sinus cavities.

Furthermore, as illustrated, the sensors 100 are positioned symmetrically. In this way, the tissue oxygenation values gathered by each of the sensors 100 are comparable to one another when reflecting the oxygenation of the right and left hemispheres. Therefore, if the two sensors 100A, 100B result in different oxygenation values, the difference can be attributed to different oxygenation of the brain hemispheres rather than different placement of the sensors 100A, 100B. This difference can alert a medical professional to medical conditions such as a hemorrhage in one hemisphere of the brain. In order to provide good care to the patient, it can be important for a medical professional to know if a difference in readings from two sensors on a patient are caused by different relative placement of the sensors or by the patient's medical condition.

In contrast, an example of improper placement of exemplary regional oximetry sensors 100A, 100B onto a patient is shown in FIG. 4. As illustrated, FIG. 4 illustrates the two sensors 100A, 100B placed at an angle measured from the horizontal axis H across the forehead A of the patient 10 that is greater than 10 degrees. For example, a first end 106 of the first sensor 100A is placed near or adjacent the patient's eyebrows, without a sufficient displacement D1 (FIG. 3) above the sinus cavities. The sensors 100A, 100B are not aligned with each other in the middle of the patient's forehead A. In this way, the light signals received by the sensors 100 may less accurately reflect brain oxygenation (for example, due to interference from sinus cavities) and be less comparable with one another. In other words, with this positioning, if different oxygenation values are obtained from the sensor 100A compared to the sensor 100B, it cannot be accurately assumed that this is due to a different oxygenation of the left and right hemispheres of the brain. Rather, this difference may be due to the improper positioning of the sensors 100A, 100B.

FIG. 5 illustrates an embodiment of a depth of consciousness sensor, illustratively the depth of consciousness sensor 200 of FIG. 2, arranged onto the forehead A of the patient 10 with proper positioning. The sensor 200 is placed above the patient's eyebrow on one side, aligning the electrodes 210 with brain tissue of the left or the right hemisphere of the brain. In some embodiments, although not shown, a second depth of consciousness sensor may be simultaneously arranged onto the forehead A of the patient such that both the left and the right hemispheres of the brain are monitored. Furthermore, FIG. 5 additionally illustrates a tail 216 of the sensor 200 which may be used to connect the sensor 200 to a monitor or other circuitry for processing signals received by the electrodes 210 of the sensors 200.

As previously described, in some instances, the physician and/or clinician may need to use multiple sensors on a patient simultaneously. For example, in some examples, both a regional oximetry sensor and a depth of consciousness sensor are arranged onto the forehead A of the patient 10 simultaneously to monitor both EEG signals and oxygen saturation values of the patient 10. The incorporation of multiple sensors requires additional coordination of placement of the sensors (e.g., regional oximetry sensor 100, depth of consciousness sensor 200) in order to ensure the sensors do not interfere with positioning of one another nor block each other from receiving the required signals. Positioning of the multiple sensors can be difficult depending on the particular procedure and the patient's anatomy and can be particularly difficult on smaller patients such as babies and children. In these instances, it may be desired to have a system and/or method for aiding the physician in placement of sensors, such as regional oximetry sensors 100 and depth of consciousness sensors 200. For example, with reference to the following figures, exemplary templates will be described that may be used for guiding proper placement of sensors onto the patient.

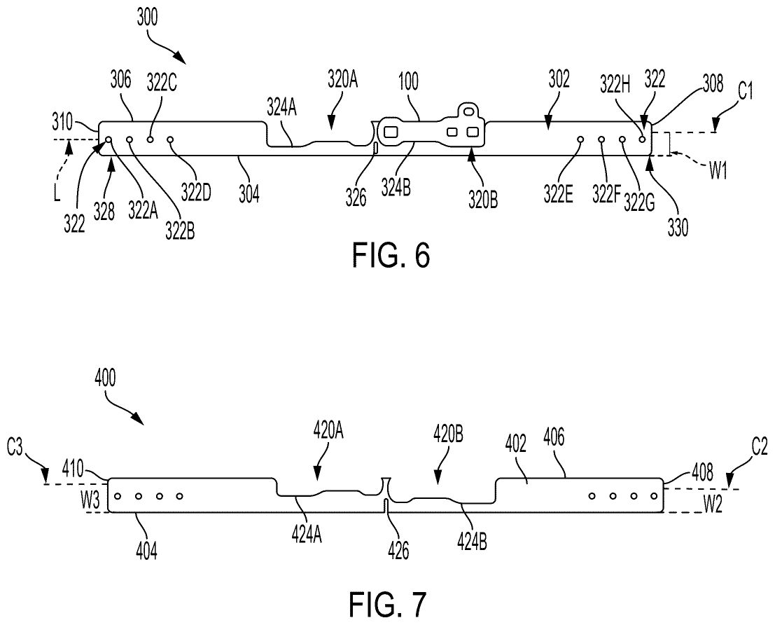

For example, FIG. 6 illustrates a sensor template 300 that may be used for guiding the placement of at least one sensor, illustratively the regional oximetry sensor 100 (shown in FIG. 1). The template 300 is sized and shaped to guide placement of the sensor 100 onto the forehead A (FIG. 3) of the patient 10 (FIG. 3). As illustrated, the template 300 includes a first surface, illustratively an upper surface 302, and a second surface (not shown) opposite the upper surface 302. The template 300 is further defined by a bottom edge 304, a top edge 306, a first side edge 308 and a second side edge 310. As illustrated, the top edge 306 is defined by at least one recess, illustratively a first recess 320A and a second recess 320B, each configured for receiving a sensor. In the example of FIG. 6, the second recess 320B is illustrated receiving the sensor 100 while the first recess 320A remains vacant. In other embodiments, the recesses 320A, 320B are shaped to accommodate other types of sensors.

With reference still to FIG. 6, each recess 320A, 320B is defined by a contour shaped to match an outer shape of the sensor 100. More specifically, each recess 320A, 320B is defined by a contoured surface. For example, the first recess 320A is defined by a first contoured surface 324A and the second recess 320B is defined by a second contoured surface 324B, each having a contour shaped to match the outer shape of the sensor 100. The first and second recesses 320A, 320B are arranged along a longitudinal axis L defined through the template 300 and may be positioned such that the recesses 320A, 320B are mirror-imaged with reference to one another and aligned with one another along a direction generally parallel to the longitudinal axis L extending through the template 300. In this way, the first recess 320A and the second recess 320B are both spaced above, and aligned with one another relative to, the bottom edge 304 of the template 300. In some instances, and as illustrated, the recesses 320A, 320B are arranged with a space extending therebetween. The space extending between the recesses 320A, 320B may be defined as a notch 326 in the template 300 that is used to position the template 300, for example by indicating a portion of the template 300 that is meant to align longitudinally with the nose of the patient 10.

As illustrated in FIG. 6, the template 300 includes a plurality of openings 322, illustratively openings 322A-322H, arranged in two groups on either end of the template 300. More particularly, openings 322A-322D are arranged along a first end 328 of the template 300, between the first end 328 and the first recess 320A. Openings 322E-322H are arranged along a second end 330 of the template 300, between the second end 330 and the second recess 320B. As will be described further herein, the openings 322 may be used to receive a suture, string or tie element for securing the first end and the second end of the template 300 with one another when the template 300 is arranged on the patient's forehead A.

Furthermore, a width W1 is defined between an axis of each recess 320A, 320B and the bottom edge 304 of the template 300. More particularly, the width W1 extends between an axis C1 extending through each recess 320A, 320B and the bottom edge 304 of the template 300. The axis C1 is arranged such that the axis C1 extends through a location corresponding to the location of the sensing elements (the optics and/or electrodes of the sensor) once the sensors are placed in the recess. In this way, the width W1 is configured such that once sensors are placed onto the forehead A of the patient 10, the optics and/or electrodes of the sensors will be arranged at the target location ensuring proper acquisition of signals from the patient. The axis C1 may or may not correspond to a central axis of the sensor, or an axis of symmetry of the sensor. In some instances, the width W1 has a value of between approximately 3 mm and approximately 25 mm. During the formation of the template 300, the width W1 may be adjusted depending on the type of sensor and/or the size of the patient that the template 300 is being used with to ensure that sufficient displacement, such as the displacement D1 of FIG. 3, is achieved once the sensors are placed onto the patient 10.

In some instances, it is advantageous for caregivers to have one template that may be used for application onto different patients and/or application of different sensor types onto the patient. This may be facilitated through a template having recesses that differ from one another in size, contoured shape, or placement within the template. For example, FIG. 7 illustrates a template 400 according to an additional embodiment of the disclosure. The template 400 includes two recesses that are offset from each other, each displaced a different amount from a bottom edge of the template. As will be described further herein, once the template 400 is placed onto a patient and properly aligned (i.e., properly placed relative to the nose and the eyebrows of the patient), the recesses define two different placements of a sensor, one of the recesses being arranged closer to the eyebrows of the patient and the other of the recesses being arranged further from the eyebrows of the patient. This arrangement defines a displacement offset between the two recesses, and more particularly between the contoured surfaces of the recesses. The two different positionings of the recesses facilitate the opportunity for two different arrangements of the sensors onto the patient. This gives the clinician two options for placing a sensor on a patient, and the clinician can use the recess that is appropriate for the particular patient. In this way, proper placement of the sensors that ensures the desired target tissue is beneath the sensor, can be achieved for patients of different sizes (i.e., an adult patient versus a child patient).

With reference still to FIG. 7, the template 400 is defined by a first surface, illustratively an upper surface 402, opposite a second surface (not shown). During use, the second surface may be configured for contact with the forehead A of the patient 10, while the upper surface 402 extends away from the forehead A. The template 400 further includes a bottom edge 404, a top edge 406, a first side edge 408 and a second side edge 410. The top edge 406 includes at least one recess, illustratively a first recess 420A and a second recess 420B. Each recess may be configured for receiving a sensor, for example the sensor 100 (FIG. 1), to guide the proper placement of the sensor 100 onto a patient. As illustrated, the second recess 420B may be similar to and/or identical to the first recess 320A of the template 300. Additionally, the template 400 may include a notch 426 defined in the bottom edge 404 of the template 400, which is situated for alignment with a physiological feature of a patient to aid in the positioning of the template 400 onto the patient. For example, the notch 426 may be configured to align with the nose of the patient to aid in the proper positioning of the template 400.

Furthermore, the first recess 420A includes a contoured surface 424A and the second recess 420B includes a contoured surface 424B, each of the contoured surfaces 424A, 424B shaped to receive a sensor. A width W2 is illustrated extending between an axis C2 of the second recess 420B and the bottom edge 404 of the template 400. Similar to the above description with reference to FIG. 6, the axis C2 of the second recess 420B is arranged such that the axis C2 extends through a location corresponding to a location of optics and/or electrodes of the sensor once the sensors are placed onto the patient 10. In some instances, the width W2 may be equal to the width W1 of the first recess 320A (FIG. 6). However, the first recess 420A may be arranged such that the contoured surface 424A of the first recess 420A is arranged further proximate to the top edge 406 than the contoured surface 424B of the second recess 420B. In this way, a width W3 defines a distance extending between an axis C3 of the first recess 420A and the bottom edge 404 of the template 400. The axis C3 is arranged such that the axis C3 extends through a location corresponding to a location of the optics and/or electrodes of the sensor once the sensors are placed onto the patient 10. In these embodiments, the width W3 is greater than the value of the width W2. In some embodiments, the width W3 may have a value of approximately 3 mm to approximately 25 mm. The values of widths W2, W3 may vary as needed such that the displacement between the patient's eyebrows and subsequently placed sensors is sufficient (i.e., equal to the desired displacement such as the displacement D1 of FIG. 3) regardless of the sensor type and anatomy of the patient. In some instances, when the template 400 is placed onto the patient 10 such that the bottom edge 404 is arranged directly above the eyebrows of the patient 10, the width W3 has a value such that a displacement between eyebrows of the patient and the sensor received within the first recess 420A is approximately 5 cm and the width W2 defined by the second recess 420B is configured such that a displacement between eyebrows of the patient and the sensor received within the second recess 420B is approximately 3 cm.

Thus, in these embodiments, the template 400 provides two placement options for the sensor 100 onto the forehead A (FIG. 3) of the patient 10 (FIG. 3) depending on the patient and the patient's anatomy. For example, the first recess 420A may be used for guiding placement of a sensor onto an adult patient while the second recess 420B may be used for guiding placement of a sensor onto a pediatric patient (e.g., a baby or child). It should be understood that the above-described configurations are provided merely as examples and the size and shape of the recesses 420A, 420B may be further modified based on the patient being monitored. For example, while the template 400 is illustrated for receiving an oximetry sensor in both recesses, in some instances, the template 400 may be modified such that one recess (e.g., the first recess) receives an oximetry sensor (e.g., sensor 100) and the other recess (e.g., the second recess) receives a depth of consciousness or EEG sensor (e.g., sensor 200).

In use, a caregiver may flip and/or rotate the template 400 to use the appropriate recess on either the left or right side of the patient. For example, the template 400 may be placed onto an adult patient and a sensor placed using the first recess 420 onto the right side of the forehead A (FIG. 3) of the patient 10 (FIG. 3) to target the tissue of the right hemisphere. The template 400 may then be flipped such that the same recess (first recess 420A) can be used on the left side of the forehead A of the patient 10 such that a sensor can be placed to target the left hemisphere of the patient 10. In this way, the same recess facilitates proper placement of two sensors onto the patient, ensuring symmetric arrangement of the sensors relative to one another and sensing of the desired target tissue.

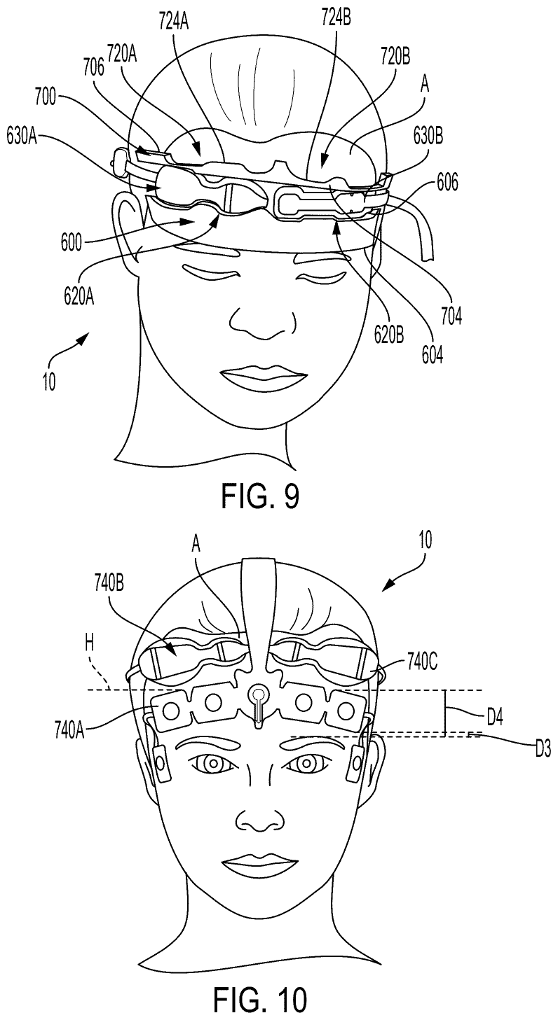

With reference now to FIGS. 8-9, exemplary use of sensor templates on a patient will be described. FIG. 8 illustrates an embodiment of a sensor template 500 arranged directly onto a patient 10. The template 500 includes a bottom edge 504 and a top edge 506 and at least two recesses, illustratively a first recess 520A and a second recess 520B, defined in the top edge 506 of the template 500. The first recess 520A is defined by a contoured surface 524A and the second recess 520B is defined by a contoured surface 524B, each of the contoured surfaces 524A, 524B being shaped to match an outline of a sensor that will be arranged within each of the recesses 520A, 520B. The template 500 is placed onto the forehead A of the patient 10 and the first recess 520A receives a first sensor 530A and the second recess 520B receives a second sensor 530B. More particularly, the first sensor 530A abuts the contoured surface 524A of the first recess 520A and the second sensor 530B abuts the contoured surface 524B of the second recess 520B. In some instances, the sensors 530A, 530B are similar to or identical to the regional oximetry sensors 100A, 100B described with reference to FIG. 1. In alternative embodiments, the first and second recesses 520A, 520B may be sized and shaped to receive depth of consciousness sensors, such as the sensors 200 (FIG. 2).

As illustrated, the template 500 is positioned with the bottom edge 504 just above the patient's eyebrows. This placement of the template 500 results in a displacement D2 defined between the top of the patient's eyebrows and a sensing axis of the sensors 530A, 530B, and more particularly, an axis of the optics of the sensors 530A, 530B. As described previously, sufficient displacement D2 for an adult patient is between approximately 3 cm and approximately 5 cm. The sufficient displacement D2 between the top of the patient's eyebrows and the sensing axis of the sensors 530A, 530B is facilitated by the value of the width W1, W2. More particularly, based on the type of sensors and physiological properties of the patient, the target value of the width W1, W2 is identified in order to ensure the sufficient displacement is achieved once the sensors are placed. Various other values of the displacement D2 may be utilized depending on the patient size and the patient anatomy. This displacement D2 provides a clinical offset of the sensor above the patient's eyebrow as needed for accurate physiological monitoring, as discussed above.

Use of the template 500 as illustrated in FIG. 8 facilitates proper placement of the sensors 530A, 530B in a similar arrangement as shown in FIG. 3. In other words, the use of the template 500 facilitates placement of the sensors 530A, 530B aligned with one another and positioned symmetrically about the forehead A of the patient 10. Furthermore, in some instances, the template 500 can be modified to include a first recess formed into the upper edge 506 of the template for a regional oximetry sensor, and a second recess formed into the bottom edge 504 of the template shaped for receiving a depth of consciousness sensor. The modified template can then assist in coordinating the placement of these two distinct types of sensors on a patient. For example, the caregiver can adjust placement of the template depending on which sensor is being placed onto the patient. For example, the caregiver can flip the template over such that the desired sensor can achieve the desired sensor placement on either side of the patient's forehead A. Other templates can accommodate additional sensors and/or additional types of sensors (EEG, temperature, capacitance, or other sensors applied to a patient's skin), and the templates can be created to account for varying patient size and anatomies.

FIG. 9 illustrates an additional exemplary embodiment of templates for use with placing sensors onto a patient. More particularly, FIG. 9 illustrates the patient 10 with two templates 600, 700 in use on the patient's forehead A, to assist in aligning multiple sensors on the patient. The first template 600 includes a bottom edge 604 and a top edge 606. The first template 600 includes a first recess 620A in the top edge 606 and a second recess 620B defined in the top edge 606 of the template 600. As illustrated, the first recess 620A is sized and shaped to receive a first sensor 630A, illustratively a first type of regional oximetry sensor. Furthermore, the second recess 620B is sized and shaped to receive a second sensor 630B, illustratively a heart rate or temperature sensor. However, in other examples, the first and second recesses 620 may be sized and shaped to receive other types of sensors.

In some instances, it may be advantageous to place additional sensors above the first set of sensors 630A, 630B. In other words, there may be a first set of sensors 630A, 630B arranged above the patient's eyebrows and a second set of sensors arranged above the first set of sensors. In these aspects, proper placement of both sets of sensors is needed to obtain accurate signals and reduce interference between the sets of sensors. In these instances, it may be desired to incorporate a second template that can be placed directly above a first set of sensors in order to properly position additional sensors. This is illustrated by the second template 700 in FIG. 9.

The second template 700 is arranged vertically above the sensors 630A, 630B. As illustrated, the second template 700 is defined by a bottom edge 704 and a top edge 706. The top edge 706 includes a plurality of recesses defined therein, including a first recess 720A and a second recess 720B. More particularly, the first recess 720A may be sized and shaped for receiving a regional oximetry sensor, such as the sensor 100 (FIG. 1), and the second recess 720B may be shaped and sized for receiving a depth of consciousness sensor, such as the sensor 200 (FIG. 2). Thus, additional sensors can be placed onto the forehead of the patient 10 while maintaining proper positioning relative to the patient's anatomy and the sensors that have already been placed onto the patient. This facilitates proper arrangement of both regional oximetry sensors and depth of consciousness sensors simultaneously. The second template 700 is used to position a second set of sensors across the patient's forehead, such that the second set of sensors are arranged just above and in some instances directly adjacent to, the first set of sensors.

For example, FIG. 10 illustrates an exemplary embodiment of a patient 10 having two rows of sensors arranged onto the forehead A. For example, FIG. 10 illustrates the patient 10 having at least three sensors positioned thereon after use of a first template and a second template. While illustrating different sensors than those shown in and discussed with reference to FIG. 9, modified templates similar to templates 600, 700 may be used for placing the two rows of sensors shown in FIG. 10.

More particularly, FIG. 10 illustrates a first row of a sensor, illustratively a first sensor 740A. The first sensor 740A is illustratively a depth of consciousness sensor (such as BIS™ bilateral sensor from Medtronic) extending across the forehead A of the patient 10. As illustrated, the first sensor 740A is arranged across both the left and right side of the patient's forehead A facilitating signal acquisition from both the left and the right hemisphere of the patient 10. However, in other instances, the first sensor 740A may be a different type of sensor, such as a regional oximetry sensor. FIG. 10 illustrates a second row of sensors arranged vertically above the first sensor 740A, the second row of sensors including a second sensor 740B and a third sensor 740C. The second sensor 740B and the third sensor 740C are each regional oximetry sensors, however, various other types of sensors may be incorporated.

The use of multiple templates for proper placement of two rows of sensors facilitates the simultaneous use and monitoring with both the regional oximetry sensors and the depth of consciousness sensor. In the illustrative example of FIG. 10, the depth of consciousness sensor (first sensor 740A) is arranged directly above the eyebrows of the patient with a displacement D3. In these instances, the displacement D3 may be defined as a distance extending between the eyebrow of the patient 10 and a sensing axis of the first sensor 740A, and more particularly, an axis extending through electrodes of the sensor 740A. During placement of the first sensor 740A, a corresponding template may be arranged directly above the eyebrows of the patient to facilitate the proper placement of and the displacement D3. In some instances, the value of D3 may range from approximately 3 cm to approximately 5 cm. Furthermore, the two regional oximetry sensors (second sensor 740B and third sensor 740C) are arranged vertically above and spaced from the first sensor 740A. The positioning of the second sensor 740B and the third sensor 740C is defined by a displacement D4 extending between a sensing axis of the second sensor 740B (or the third sensor 740C) and the eyebrow of the patient 10. The sensing axis of the second sensor 740B (or the third sensor 740C) may be defined as the axis that passes through the optics of the second sensor 740B (or the third sensor 740C). The value of displacement D4 is dependent on the thickness of the first row of sensors (the first sensor 740A) and the displacement D3. In some instances, the displacement D4 is greater than 3 cm. In further embodiments, the displacement is greater than 5 cm. The above described embodiment is presented as an exemplary embodiment, and is not meant to be limiting. For example, in other examples, the regional oximetry sensors (second sensor 740B and third sensor 740C) may be arranged directly adjacent one another and vertically below the depth of consciousness sensor (first sensor 740A). In further examples, more than three sensors may be arranged onto the forehead A of the patient 10 and different types of sensors may be incorporated.

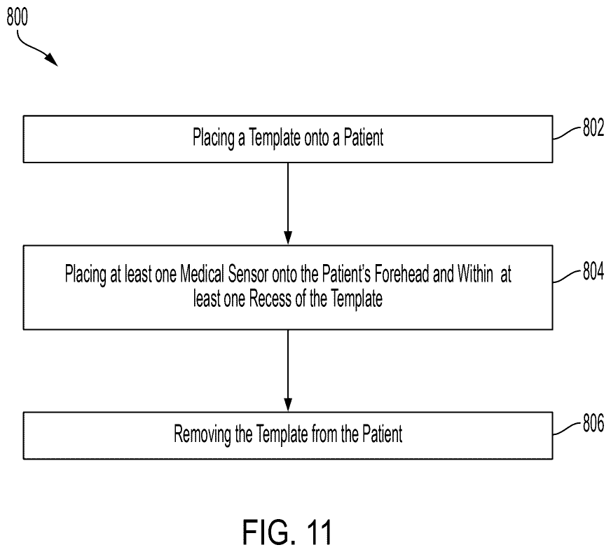

With reference now to FIG. 11, a method 800 for applying medical sensors onto a patient is described. More particularly, the method 800 will be described with reference to applying the medical sensors 100A, 100B (FIG. 1), onto the forehead A of the patient 10 using the template 300 (FIG. 6). However, in other examples, the method 800 may be used with any of the sensors and templates described herein.

The method 800 first includes step 802 of placing a template (for example, the template 300) onto the patient, and more particularly above the eyebrows of the patient. Placing the template 300 may include aligning a structural or visible feature of the template (such as the notch 326) with an anatomical feature of the patient (such as the nose of the patient) to facilitate proper positioning of the template relative to the anatomy of the patient. In some instances, the step 802 may further include placing the template onto the forehead and using a fixation element (string, sutures, etc.) to tie the first end (such as the end 302) of the template with the second end (such as end 304) of the template around the back of the patient's head to secure the template to the patient. In other instances, the template may be temporarily secured to the patient with an alternative method or mechanism such as a temporary adhesive or fixation mechanism.

At step 804, the method 800 includes placing at least one medical sensor onto the forehead of the patient, at step 804, within at least one recess of the template. For example, this may include placing the first sensor 100A (FIG. 1) into the first recess 320A (FIG. 3) and the second sensor 100B (FIG. 1) into the second recess 320B (FIG. 3) of the template 300. This includes placing the sensors 100A, 100B against the countered surfaces 324A, 324B of the recesses 320A, 320B, respectively. Furthermore, in some instances, the step 804 includes adhering the sensors onto the patient's forehead. For example, as previously noted, the lower surface 102 of the sensors 100A, 100B can be a surface of adhesive and/or glue so that when the sensors 100 are placed onto the forehead A, the sensors 100A, 100B are inherently adhered with the sensors 100.

As illustrated, the method 800 includes the step 806 of removing the template from the patient. Once the sensors are properly positioned, the template is no longer needed, and the template can be removed from the patient. In some instances, this includes severing the attachment of the first end and the second end of the template so that the template can be easily removed. In other embodiments, if the template is otherwise attached or adhered to the patient's skin, the template can be peeled off of the skin of the patient. After removal, the template can be cleaned and sterilized and reused for application of one or more sensors onto an additional patient. The template can undergo the application onto a patient, removal from a patient, cleaning and sterilization, and reapplication onto another patient more than once, and in some examples, any number of times until degradation of the template.

In some embodiments, the method 800 can further include applying a second template (for example, template 700 of FIG. 9) onto the patient's forehead such that an additional set of sensors can be applied onto the patient. In some cases, this includes applying depth of consciousness sensors (for example, sensor 200 of FIG. 2) onto the patient. In these instances, once the additional set of sensors is arranged onto the patient, the second template may be removed from the patient prior to patient monitoring.

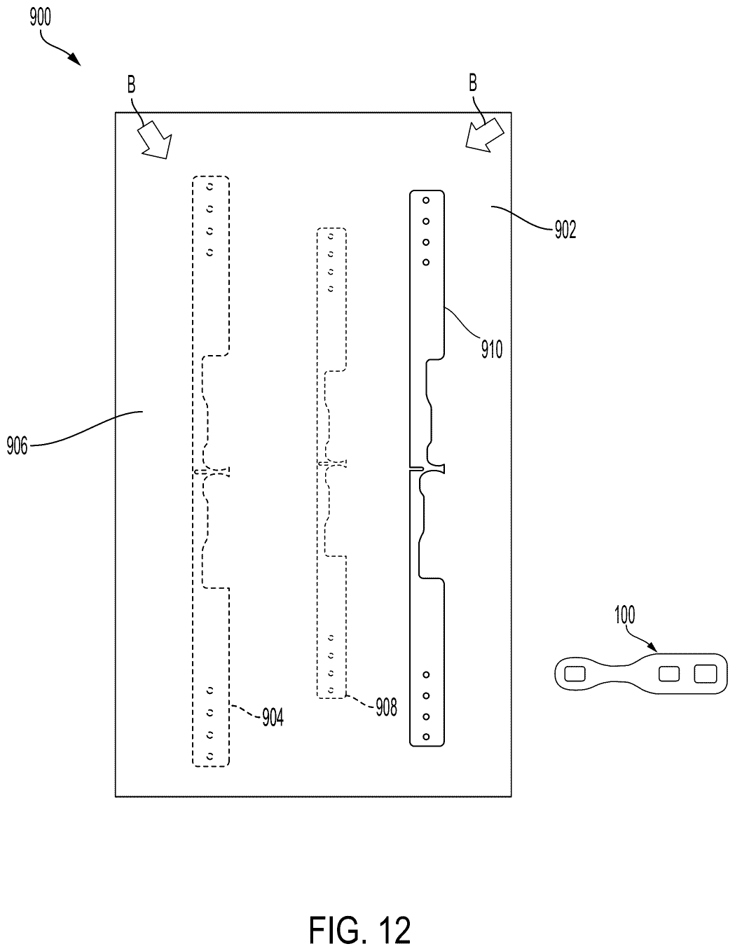

In some embodiments, the steps of method 800 can be completed by a medical caregiver using a kit of materials provided to the physician. For example, FIG. 12 illustrates an exemplary embodiment of a kit 900 for use by a medical caregiver in placing medical sensors onto a patient. In some embodiments, the kit 900 may include a template and at least one medical sensor enclosed within a single package. The kit 900 includes a package, a medical sensor, and a template with a recess that matches a contour of the medical sensor. The kit may include more than one medical sensor and/or more than one template.

In other embodiments, the packaging of the sensor defines the template. For example, as illustrated in FIG. 12, the kit 900 includes a package 902 having an outer surface 906 enclosing the sensor, illustratively the regional oximetry sensor 100, and a first template 904 embedded in a surface of the package 902. While the sensor 100 is illustrated as external to the package 902 in FIG. 12, this is for clarity and it should be understood the sensor 100 is arranged inside of the package 902 of the kit 900.

The outer surface 906 is configured to be peeled away or removed from the package 902 (as indicated by arrows B). An outline of the template 904 is embedded (such as imprinted, ink printed or perforated) onto the outer surface 906 of the package 902 such that a caregiver can cut or tear the template 904 directly from the outer surface 906 of the package 902. In the illustrative embodiment of FIG. 12, the broken line of the outline of the template 904 may indicate that the template 904 is a perforation in the outer surface 906 such that the caregiver can tear the template 904 directly from the outer surface 906. The template 904 may then be used by the physician to guide the placement of the sensors onto a patient, for example with the method 800 of FIG. 8. In some embodiments, the template 904 embedded onto the package 902 may include the outline of several different templates and/or types of templates, and a physician may choose which template is most applicable to the desired use (sensor type, arrangement, patient anatomy) for use during monitoring. For example, the outer surface 906 includes an outline of a second template 908 embedded onto the package 902. As illustrated, the second template 908 is smaller in size than the first template 904 and thus may be used by the caregiver when appropriate depending on the size and the age of the patient. In these instances, the second template 908 may also be indicated as a perforation in the package 902, as indicated by the broken lines. In these instances, the package 902 additionally includes a third template 910 embedded onto the outer surface 906 of the package 902. As indicated by the solid outline of the template 910, the template 910 may be ink-printed or otherwise drawn onto the package 902 and configured to be directly cut by the caregiver prior to placement onto the patient. The third template 910 may be similar to and/or the same as the template 400, discussed with reference to FIG. 7, and thus may facilitate the placement of different types of sensors onto the patient.

In further embodiments, the kit 900 may include any number of sensors and templates arranged within the packaging. The kit 900 may include any number of sensors arranging within the packaging and more than one, two, or three template outlines may be defined into the surface of the package. The illustrative example of the kit 900 is provided merely as an example and additional sensors, templates, or other components may be incorporated.

As described herein, use of the one or more templates results in placement of dual medical sensors in a way that is symmetric, parallel to the eyebrows, aligned in the middle, and displaced above the eyebrows. The use of one or more templates facilitates proper placement of medical sensors to allow for proper simultaneous monitoring of the patients with different types of medical sensors. Furthermore, the one or more templates facilitate proper anatomical placement of the medical sensors on the patient. In further embodiments, use of two templates results in placement of three, four, or more medical sensors in appropriate configurations on the patient.

In Aspect 1, a guide for placement of a medical sensor onto a patient includes a template comprising an upper edge, a lower edge, and a recess defined in the upper edge of the template, the recess comprising a contour that matches a shape of a medical sensor such that the recess is sized to seat the medical sensor into the recess, wherein the recess is displaced above the lower edge of the template by a defined distance corresponding to a clinical offset of the medical sensor above a patient's eyebrow.

Aspect 2 includes the guide of Aspect 1, where the recess comprises a first recess and wherein the template further comprises a second recess, each of the first and second recess defined in the upper edge of the template.

Aspect 3 includes the guide of Aspect 2, wherein the template includes a notch arranged between the first recess and the second recess and oriented to align with an anatomical feature of the patient to guide placement of the template onto the patient.

Aspect 4 includes the guide of Aspect 1, 2, or 3, wherein the template comprises a first end opposite a second end, and the first end and the second end include a plurality of openings positioned along the template for securing the template onto a patient.

Aspect 5 includes the guide of any preceding Aspect, wherein the medical sensor comprises a regional oximetry sensor.

Aspect 6 includes the guide of any preceding Aspect, wherein the medical sensor comprises a depth of consciousness sensor.

Aspect 7 includes the guide of Aspect 2, wherein the defined distance comprises a first distance, and wherein the second recess is displaced from the lower edge of the template by a second distance, and wherein the first distance is less than the second distance.

Aspect 8 includes the guide of Aspect 7, wherein the first distance comprises a value of between approximately 3 mm to approximately 25 mm.

In Aspect 9 a kit for placement of at least one medical sensor includes a package having an outer surface; a template including an upper edge, a lower edge, and at least one recess defined in

the upper edge and comprising a contour; and a medical sensor inside the package, at least a portion of the medical sensor having a shape that matches the contour.

Aspect 10 includes the guide of Aspect 9, wherein the template is embedded into the outer surface of the package.

Aspect 11 includes the guide of Aspect 9 or 10, wherein the medical sensor comprises a first medical sensor and wherein the kit further comprises a second medical sensor.

Aspect 12 includes the guide of Aspect 11, wherein the first medical sensor and the second medical sensor comprise regional oximetry sensors.

Aspect 13 includes the guide of Aspect 11, wherein the first medical sensor and the second medical sensor comprise depth of consciousness sensors.

Aspect 14 includes the guide of Aspect 9, 10, 11, 12, or 13, further comprising a second template, wherein the first template is larger than the second template

Aspect 15 includes the guide of Aspect 9, 10, 11, 12, 13, or 14, further comprising a second template, wherein the second template comprises a lower edge that matches a shape of the at least one medical sensor such that the second template aligns above the at least one medical sensor in placement on the patient.

In Aspect 16, a guide for placement of a medical sensor onto a patient includes a template comprising an upper edge and a lower edge; a first recess and a second recess defined in the upper edge of the template and mirror-imaged with each other, the first and second recesses each comprising a contour that matches a shape of a medical sensor, wherein the first recess is displaced above the lower edge of the template by a first distance and wherein the second recess is displaced above the lower edge of the template by a second distance.

Aspect 17 includes the guide of Aspect 16, wherein the first and second distances are the same.

Aspect 18 includes the guide of Aspect 16, wherein the first distance corresponds to a first clinical offset of the medical sensor above a patient's eyebrow for a pediatric patient, and wherein the second distance corresponds to a second clinical offset of the medical sensor above a patient's eyebrow for an adult patient.

Aspect 19 includes the guide of Aspect 16, 17, or 18, wherein the contour matches a regional oximetry sensor.

Aspect 20 includes the guide of Aspect 16, 17, 18, or 19, wherein the template is defined in a package of the medical sensor.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A guide for placement of a medical sensor onto a patient, the guide comprising:
a template comprising an upper edge, a lower edge, and a recess defined in the upper edge of the template, the recess comprising a contour that matches a shape of a medical sensor such that the recess is sized to seat the medical sensor into the recess,
wherein the recess is displaced above the lower edge of the template by a defined distance corresponding to a clinical offset of the medical sensor above a patient's eyebrow.

2. The guide of claim 1, where the recess comprises a first recess and wherein the template further comprises a second recess, each of the first and second recess defined in the upper edge of the template.

3. The guide of claim 2, wherein the template includes a notch arranged between the first recess and the second recess and oriented to align with an anatomical feature of the patient to guide placement of the template onto the patient.

4. The guide of claim 1, wherein the template comprises a first end opposite a second end, and the first end and the second end include a plurality of openings positioned along the template for securing the template onto a patient.

5. The guide of claim 1, wherein the medical sensor comprises a regional oximetry sensor.

6. The guide of claim 1, wherein the medical sensor comprises a depth of consciousness sensor.

7. The guide of claim 2, wherein the defined distance comprises a first distance, and wherein the second recess is displaced from the lower edge of the template by a second distance, and wherein the first distance is less than the second distance.

8. The guide of claim 7, wherein the first distance comprises a value of between approximately 3 mm to approximately 25 mm.

9. A kit for placement of at least one medical sensor, the kit comprising:
a package having an outer surface;
a template including an upper edge, a lower edge, and at least one recess defined in the upper edge and comprising a contour; and
a medical sensor inside the package, at least a portion of the medical sensor having a shape that matches the contour.

10. The kit of claim 9, wherein the template is embedded into the outer surface of the package.

11. The kit of claim 9, wherein the medical sensor comprises a first medical sensor and wherein the kit further comprises a second medical sensor.

12. The kit of claim 11, wherein the first medical sensor and the second medical sensor comprise regional oximetry sensors.

13. The kit of claim 11, wherein the first medical sensor and the second medical sensor comprise depth of consciousness sensors.

14. The kit of claim 9, further comprising a second template, wherein the first template is larger than the second template.

15. The kit of claim 9, further comprising a second template, wherein the second template comprises a lower edge that matches a shape of the at least one medical sensor such that the second template aligns above the at least one medical sensor in placement on the patient.

16. A guide for placement of a medical sensor onto a patient, the guide comprising:
a template comprising an upper edge and a lower edge;
a first recess and a second recess defined in the upper edge of the template and mirror-imaged with each other, the first and second recesses each comprising a contour that matches a shape of a medical sensor,
wherein the first recess is displaced above the lower edge of the template by a first distance and wherein the second recess is displaced above the lower edge of the template by a second distance.

17. The guide of claim 16, wherein the first and second distances are the same.

18. The guide of claim 16, wherein the first distance corresponds to a first clinical offset of the medical sensor above a patient's eyebrow for a pediatric patient, and wherein the second distance corresponds to a second clinical offset of the medical sensor above a patient's eyebrow for an adult patient.

19. The guide of claim 16, wherein the contour matches a regional oximetry sensor.

20. The guide of claim 16, wherein the template is defined in a package of the medical sensor.
